(19) United States
(12) Patent Application Publication
(10) Pub. No.: US 2023/0145939 A1
(43) Pub. Date: May 11, 2023

(54) MARKER COMPOSITION FOR CANCER DIAGNOSIS OR PROGNOSIS BASED ON EXOSOME OVEREXRESSING GCC2

(71) Applicant: Korea University Research and Business Foundation, Seoul (KR)

(72) Inventors: Hyun Koo Kim, Seoul (KR); Byeong Hyeon Choi, Seoul (KR); Yuhua Quan, Seoul (KR); Jiyun Rho, Incheon (KR)

(21) Appl. No.: 17/918,492

(22) PCT Filed: Apr. 5, 2021
(86) PCT No.: PCT/KR2021/004187

§ 371 Date: Oct. 12, 2022

(30) Foreign Application Priority Data
Apr. 13, 2020 (KR) 10-2020-0044608

(51) Int. Cl. C12Q 1/6886; G01N 33/574

(52) U.S. Cl. CPC C12Q 1/6886; G01N 33/57484; G01N 2500/04; G01N 2800/52; C12Q 2600/118; C12Q 2600/136; C12Q 2600/158

(57) ABSTRACT

Provided according to one embodiment of the present invention is a marker composition for diagnosis or prognosis of esophageal cancer or thymic carcinoma, comprising an exosome overexpressing GRIP and coiled-coil domain-containing protein 2 (GCC2).

### TECHNICAL FIELD

The following description relates to a marker composition for cancer diagnosis or prognosis prediction based on an exosome overexpressing GCC2 and more particularly, to a marker composition for diagnosis or prognosis prediction of esophageal cancer or thymic cancer.

### BACKGROUND ART

A tumor is a result of uncontrolled and disordered cell proliferation caused by an excess of abnormal cells, and if such a tumor has destructive proliferation, invasion, and metastasis, it is classified as a malignant tumor, that is, cancer.

Currently, examination means for diagnosing cancer include X-ray imaging, endoscopy, biopsy, and the like. However, although the examination process of these methods is relatively simple, these methods have problems that the diagnosis success rate is not high, and problems in hygiene and patient suffering in the course of the examination. Therefore, there is a need for a method for diagnosing cancer to replace it.

In order to treat cancer, it is important to diagnose cancer with high sensitivity and specificity in the pre-treatment stage, and a high cure rate can be achieved only when cancer is detected at an early stage through such diagnosis.

Therefore, there is a need for the development of a non-invasive, high-sensitivity and high-specification method capable of diagnosing cancer at an early stage. Until now, molecular diagnostic technology for diagnosing cancer by specifically detecting a lesion at an early stage is insignificant, and furthermore, there is no method specifically applied to specific cancer.

### PRIOR ART LITERATURE

Patent document: KR Patent Publication No. 10-2015-0087580

### DISCLOSURE OF THE INVENTION

### Technical Goals

An object of the present invention is to provide a marker composition capable of improving the accuracy of diagnosis of esophageal cancer while being able to use it in a non-invasive manner and a marker composition for diagnosis or prognosis prediction of esophageal cancer or thymic cancer, including an exosome overexpressing GRIP and coiled-coil domain-containing protein (GCC2).

Another object of the present invention is to provide a method for providing information necessary for diagnosing esophageal cancer or pancreatic cancer or predicting a prognosis thereof, the method including steps of: (a) separating an exosome from a biological sample isolated from a subject; and (b) measuring the expression level of a GRIP and coiled-coil domain-containing protein (GCC2) gene or protein in the exosome.

However, the task to be achieved by the present invention are not limited to the tasks mentioned above, and other tasks not mentioned are clearly understood by those of ordinary skill in the art from the following description.

### Technical Solutions

According to an example, the present invention provides a marker composition for diagnosing or predicting prognosis of esophageal or thymic cancer, the composition including an exosome overexpressing a GRIP and coiled-coil domain-containing protein (GCC2) protein.

According to an example, the present invention provides a composition for diagnosing or predicting prognosis of esophageal or thymic cancer, the composition including a primer or probe that specifically binds to a GRIP and coiled-coil domain-containing protein (GCC2) gene in an exosome.

According to an example, the present invention provides a composition for diagnosing or predicting prognosis of esophageal or thymic cancer, the composition including an antibody that specifically binds to a GRIP and coiled-coil domain-containing protein (GCC2) in an exosome.

According to an example, the present invention provides a kit for diagnosing or predicting prognosis of esophageal cancer or thymic cancer, the kit including the composition.

According to an aspect, the kit may include at least one selected from the group consisting of an RT-PCR kit, a microarray chip kit, a DNA kit and a protein chip kit.

According to an example, the present invention provides a method for providing information necessary for diagnosing esophageal cancer or predicting a prognosis thereof, the method including steps of: (a) separating an exosome from a biological sample isolated from a subject; and (b) measuring the expression level of a GRIP and coiled-coil domain-containing protein (GCC2) gene or protein in the exosome.

According to an aspect, the biological sample may include at least one selected from the group consisting of whole blood, serum, plasma, saliva, urine, sputum, lymph and cells.

According to an aspect, the method may further include a step of comparing the measured expression level of the GCC2 gene or protein with a predetermined cutoff value, and the predetermined cutoff value may be 521.9695 pg/ml.

According to another example, the present invention provides a method for providing information necessary for diagnosing thymic cancer or predicting a prognosis thereof, the method including steps of: (a) separating an exosome from a biological sample isolated from a subject; and (b) measuring the expression level of a GRIP and coiled-coil domain-containing protein (GCC2) gene or protein in the exosome.

According to an aspect, the biological sample may include at least one selected from the group consisting of whole blood, serum, plasma, saliva, urine, sputum, lymph and cells.

According to an aspect, the method may further include a step of comparing the measured expression level of the GCC2 gene or protein with a predetermined cutoff value, and the predetermined cutoff value may be 909.4513 pg/ml.

According to still another example, the present invention provides a method for screening a therapeutic agent for esophageal cancer, the method including steps of: (a) treating a candidate substance for the therapeutic agent for esophageal cancer in a biological sample isolated from a patient with esophageal cancer; (b) separating an exosome from the biological sample; and (c) measuring the expression level of a GRIP and coiled-coil domain-containing protein (GCC2) gene or protein in the exosome.

According to yet another example, the present invention provides a method for screening a therapeutic agent for thymic cancer, the method including steps of: (a) treating a candidate substance for the therapeutic agent for thymic cancer in a biological sample isolated from a patient with thymic cancer; (b) separating an exosome from the biological sample; and (c) measuring the expression level of a GRIP and coiled-coil domain-containing protein (GCC2) gene or protein in the exosome.

### Advantageous Effects

The marker composition of the present invention includes genes or proteins overexpressed in exosomes of esophageal cancer patients or thymic cancer patients to non-invasively and highly accurately diagnose esophageal cancer or thymic cancer or predict the prognosis by measuring its expression level.

It should be understood that the effects of the present invention are not limited to the above-described effects and include all effects that can be inferred from the configuration of the invention described in the detailed description or claims of the present invention.

### BRIEF DESCRIPTION OF DRAWINGS

FIG. 1 is a view showing a standard curve for quantifying exosomes extracted from esophageal cell lines and esophageal cancer cell lines.

FIG. 2 is a view showing a comparison of the protein composition in the exosomes extracted from the esophageal cell line and esophageal cancer cell line.

FIG. 3 is a view showing the results of ELISA analysis comparing the expression level of GCC2 protein in the blood exosomes of normal people and esophageal cancer patients.

FIG. 4 is a view showing the ROC curve for deriving the cutoff value of the GCC2 protein concentration in the blood exosomes of normal people and esophageal cancer patients.

FIG. 5 is a view showing the results of ELISA analysis comparing the expression level of GCC2 protein in the blood exosomes of normal people and thymic cancer patients.

FIG. 6 is a view showing the ROC curve for deriving the cutoff value of the GCC2 protein concentration in the blood exosomes of normal people and thymic cancer patients.

### BEST MODE FOR CARRYING OUT THE INVENTION

Hereinafter, examples will be described in detail with reference to the accompanying drawings. Like reference numerals in each figure indicate like elements.

Various modifications may be made to the examples described below. It should be understood that the examples described below are not intended to limit an embodiment and include all modifications, equivalents, and substitutions thereto.

Terms used in the examples are used only to describe specific examples and are not intended to limit the examples. The singular expression includes the plural expression unless the context clearly dictates otherwise. It should be understood that in the present specification, terms such as “comprise” or “have” are intended to designate that a feature, number, step, operation, component, part, or a combination thereof described in the specification exists, but the possibility of the presence or addition of one or more other features, numbers, steps, operations, components, parts, or combinations thereof are not precluded in advance.

Unless defined otherwise, all terms used herein, including technical or scientific terms, have the same meaning as commonly understood by one of ordinary skill in the art to which the example belongs. Terms such as those defined in a commonly used dictionary should be interpreted as having a meaning consistent with the meaning in the context of the related art, and should not be interpreted in an ideal or excessively formal meaning unless explicitly defined in the present application.

In addition, in the description with reference to the accompanying drawings, the same components are assigned the same reference numerals regardless of the reference numerals, and the overlapping description thereof is excluded. In describing the example, if it is determined that a detailed description of a related known technology may unnecessarily obscure the gist of the example, the detailed description thereof is excluded.

According to one example, the present invention provides a marker composition for diagnosing or predicting prognosis of esophageal or thymic cancer, the composition including an exosome overexpressing a GRIP and coiled-coil domain-containing protein (GCC2) gene or protein.

As used herein, the term “GCC2 gene or protein-overexpressing exosome” refers to an exosome expressing a GCC2 gene or protein at a high level compared to the GCC2 expression level or a predetermined cutoff value of the exosome isolated from a normal person.

An exosome is a small endoplasmic reticulum of nano size (30-150 nm) secreted by most cells. It is known that various types of proteins, genetic materials (DNA, mRNA, or miRNA), lipids, etc. derived from cells are contained in the exosome inner and phospholipid double lateral membranes. In addition, it has been reported that tissue-derived exosomes can be used for the diagnosis of diseases because they reflect the state of the tissue that secreted them.

Accordingly, the present inventors have completed the present invention by confirming that the GCC2 gene or protein specifically expressed in exosomes derived from esophageal cancer and thymic cancer is used to accurately and quickly diagnose cancer or predict the prognosis thereof.

As used herein, the term “diagnosis” refers to determining the presence or characteristics of a pathological condition, that is, whether esophageal cancer or thymic cancer develops. In addition, “prognosis” refers to determining whether the subject has relapses, metastases, drug reactivity, or resistance after treatment for esophageal cancer or thymic cancer. This may include the concept of predicting whether or not the subject will develop esophageal cancer as well as predicting whether the subject's survival prognosis will be good in the future by measuring the expression level of GCC2 in exosomes isolated from the subject's sample.

Since it is possible to diagnose or predict the prognosis of esophageal cancer or thymic cancer by measuring the expression level of the GCC2 gene or protein, a primer or probe that specifically binds to its gene, or an antibody that specifically binds to its protein may be used for a composition for diagnosing or predicting prognosis of esophageal cancer or thymic cancer.

In addition, a primer or probe that specifically binds to the GCC2 gene, or antibody that specifically binds to the GCC2 protein is applied to the kit to provide a kit for diagnosing or predicting prognosis of esophageal cancer or thymic cancer.

The kit may include an RT-PCR kit, a microarray chip kit, a DNA kit, a protein chip kit, and the like but is not limited thereto. The kit can confirm and detect the expression level of the GCC2 gene or protein corresponding to the marker in the exosome to diagnose or predict esophageal cancer.

The kit may include primers, probes, or antibodies that selectively recognize markers for diagnosis or prediction of prognosis of esophageal cancer or thymic cancer, as well as one or more other component compositions, solutions, or devices suitable for analysis methods.

In addition, the kit may include a substrate, a suitable buffer solution, a secondary antibody labeled with a chromogenic enzyme or a fluorescent substance, and a chromogenic substrate for immunological detection of the antibody. As the substrate, a nitrocellulose membrane, a 96-well plate synthesized from a polyvinyl resin, a 96-well plate synthesized from a polystyrene resin, and a glass slide glass may be used.

Peroxidase, alkaline phosphatase, etc. may be used as a chromogenic enzyme. FITC, RITC, etc. may be used as a fluorescent material. ABTS (2,2′-azino-bis-(3-ethylbenzothiazoline-6-sulfonic acid), OPD (0-phenylenediamine), or TMB (tetramethyl benzidine) may be used as a chromogenic substrate solution.

According to still another example, the present invention provides a method for providing information necessary for diagnosing esophageal cancer or predicting a prognosis thereof, the method including steps of: (a) separating an exosome from a biological sample isolated from a subject; and (b) measuring the expression level of a GRIP and coiled-coil domain-containing protein (GCC2) gene or protein in the exosome.

In addition, according to still another example, the present invention provides a method for providing information necessary for diagnosing thymic cancer or predicting a prognosis thereof, the method including steps of: (a) separating an exosome from a biological sample isolated from a subject; and (b) measuring the expression level of a GRIP and coiled-coil domain-containing protein (GCC2) gene or protein in the exosome.

The biological sample may be one or more selected from the group consisting of whole blood, serum, plasma, saliva, urine, sputum, lymph, and cells, preferably whole blood or cells, but is not limited thereto.

The gene expression level measurement is a process of determining the presence and expression level of the mRNA of the GCC2 gene from a biological sample for diagnosing or predicting the prognosis of esophageal cancer and means measuring the mRNA expression level.

Analytical methods for this include reverse transcription polymerase reaction (RT-PCR), competitive reverse transcription polymerase reaction (competitive RT-PCR), real-time reverse transcription polymerase reaction (real-time RT-PCR), RNase protection assay (RPA), northern blotting, a DNA chip, and the like, but is not limited thereto.

In addition, the protein expression level measurement refers to a process of confirming the presence and expression level of GCC2 protein from a biological sample for diagnosing or predicting prognosis of esophageal or thymic cancer.

The measurement or comparison of protein expression level may be achieved using a method well known in the art, examples of which include, but are not limited to, protein chip assay, immunoassay, ligand binding assay, matrix assisted laser desorption/ionization time of flight mass spectrometry (MALDI-TOF), surface enhanced laser desorption/ionization time of flight mass spectrometry (SELDI-TOF), radioimmunoassay, radial immunodiffusion, Ouchterlony immunodiffusion, Rocket immunoelectrophoresis, immunohistostaining, complement fixation test, 2-D electrophoresis, liquid chromatography-mass spectrometry (LC-MS), liquid chromatography-mass spectrometry/mass spectrometry (LC-MS/MS), Western blotting, and ELISA (enzyme-linked immunosorbentassay), but is not limited thereto.

The method for providing information of the present invention may further include a step of comparing the measured gene or protein expression level of GCC2 with the expression level of the GCC2 gene or protein of a normal control or a predetermined cutoff value.

When the expression level of the GCC2 gene or protein in the exosome of the subject is higher than that of the normal control or higher than the predetermined cutoff value of 521.9695 pg/ml, it can be determined that esophageal cancer has occurred or has a high probability of occurrence, or when it is higher than the predetermined cutoff value of 909.4513 pg/ml, it can be determined that thymic cancer has occurred or has a high probability of occurrence.

According to an example, the present invention provides a method for screening a therapeutic agent for esophageal cancer, the method including steps of: (a) treating a candidate substance for the therapeutic agent for esophageal cancer in a biological sample isolated from a patient with esophageal cancer; (b) separating an exosome from the biological sample; and (c) measuring the expression level of a GRIP and coiled-coil domain-containing protein (GCC2) gene or protein in the exosome.

In addition, the present invention provides a method for screening a therapeutic agent for thymic cancer, the method including steps of: (a) treating a candidate substance for the therapeutic agent for thymic cancer in a biological sample isolated from a patient with thymic cancer; (b) separating an exosome from the biological sample; and (c) measuring the expression level of a GRIP and coiled-coil domain-containing protein (GCC2) gene or protein in the exosome.

As an extension of the method for providing information necessary for diagnosing or predicting prognosis of esophageal cancer or thymic cancer, the screening for candidate substances for the therapeutic agent may be applied. That is, when a biological sample isolated from an esophageal cancer patient or a thymic cancer patient were treated with a candidate substance for treating esophageal cancer or thymic cancer, and a decrease in the expression level of the GCC2 gene or protein is confirmed in the exosomes present therein, it may be inferred that the candidate substance effectively functions as a therapeutic agent for esophageal cancer.

Hereinafter, the present invention is described in more detail through examples. The following examples are described for the purpose of illustrating the present invention, but the scope of the present invention is not limited thereto.

### Example 1: Comparison of GCC2 in Exosomes of Normal Cells and Esophageal Cancer Cells

Three types of esophageal (cancer) cell lines (HET1A, TE8, and TE12), respectively, were cultured in 150 mm diameter dishes. At this time, an ultra-high speed centrifuge was used to perform centrifugation at 120,000 g for 4 hours, and the supernatant of fetal bovine serum (FBS) from which exosomes were removed was used as a culture medium. Using the culture medium, cells were continuously cultured for 2 to 3 days to reach 70 to 80% confluency. The obtained culture medium was centrifuged at 10,000 g for 30 minutes to remove cell debris and sequentially passed through 0.45 μm and 0.22 μm filters to preferentially remove relatively bulky materials. The filtered cell culture medium was concentrated using an Amicon tube 100 K (Millipore, USA), leaving only particles of the desired size.

Next, only the particles of the exosome size (50-250 nm) were separated from the concentrated cell culture medium using size exclusion chromatography and concentrated again using an Amicon tube 100 K. RIPA lysis buffer (Thermo Fisher Scientific, USA) was used to obtain proteins from the concentrated exosomes, and western blotting was performed to confirm the expression level of GCC2 protein in the obtained exosomes.

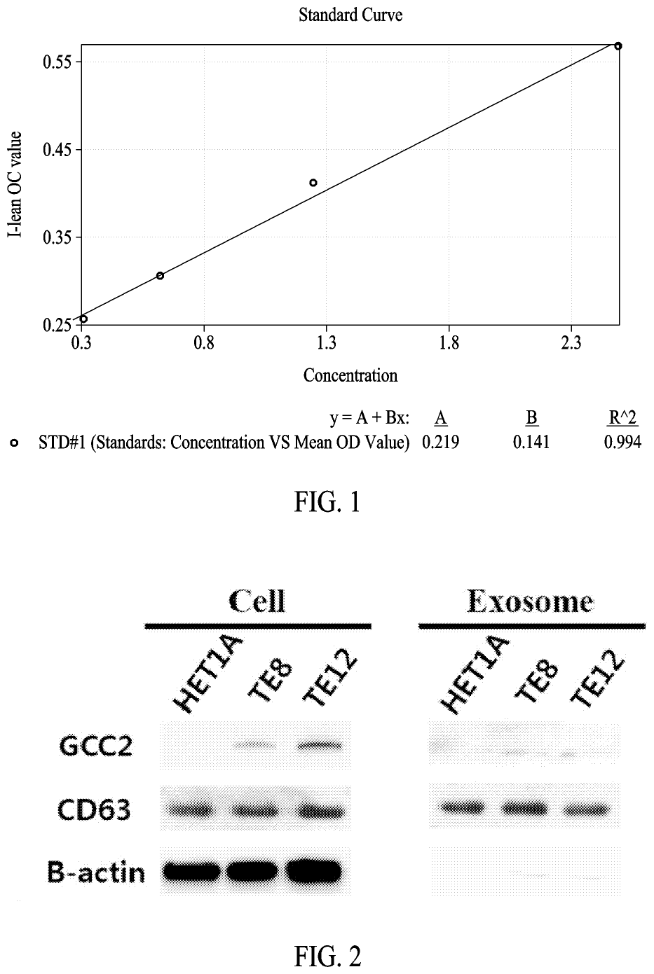

Specifically, exosomes were extracted from normal esophageal cell lines and esophageal cancer cell lines using a RIPA lysis buffer containing a protease inhibitor cocktail, and both exosomes were lysed to obtain a protein. The obtained protein was quantified using the Bradford assay method, and a standard curve was obtained (FIG. 1). The equation was y=0.219+0.141x, and the R-square value was calculated to be 0.994. The OD value of the protein obtained from the exosome was measured at a wavelength of 595 nm and is shown in Table 1 below.

TABLE 1

| Classification | HET1A | TE8 | TE12 |
|---|---|---|---|
| OD value | 0.4 | 0.53 | 0.61 |

The concentrations of HET1A, TE8, and TE12 were confirmed to be 1243 μg/ml, 2213 μg/ml, and 2766 μg/ml, respectively, and based on this, 30 μg of protein obtained from cells and 30 μg of protein obtained from exosomes were inactivated with heat to prepare samples. The total protein lysate was loaded into each lane on 10% SDS-PAGE gel and separated by size. The separated protein was attached to the nitrocellulose membrane and blocked with Tris-buffered saline-Tween 20 containing 5% skim milk. The nitrocellulose membrane was treated with primary antibodies GCC2 (1:500, Santa Cruz), CD63 (1:500, Santa Cruz), and beta-actin (1:1000, Santa Cruz) overnight at 4° C. Then, after treatment with anti-goat IgG-HRP, anti-mouse IgG-HRP, and anti-rabbit IgG-HRP-binding secondary antibody, it was washed and reacted with ECL buffer (Bio-rad, USA), and an image obtained using the FluorChem E system (proteinsimple, USA) is shown in FIG. 2.

Referring to FIG. 2, it can be seen that the protein composition ratio of normal esophageal cells and esophageal cancer cells is different from each other in the same amount of exosome protein. That is, it can be seen that cancer cells have a different protein composition from normal cells, and GCC2 is strongly expressed in exosomes derived from esophageal cancer cells, unlike normal esophageal cells.

### Example 2: Measurement of GCC2 Protein Expression Level in Exosome Derived from Esophageal Cancer Patient

Plasma from an esophageal cancer patient was centrifuged at 4° C. and 5,000×g for 15 minutes to remove debris present in the plasma sample. 500 ul of plasma samples were taken, and exosomes were extracted through size exclusion column chromatography.

In order to confirm whether exosomes containing GCC2 protein are available as markers for diagnosing or predicting prognosis of esophageal cancer, ELISA assay was performed using Mybiosource's GRIP and coiled-coil domain containing protein 2 ELISA KIT according to the manufacturer's protocol.

Specifically, two standard samples, two blank samples, and two exosome samples extracted in Example 1 were prepared, and 50 ul each was injected into a 96-well ELISA plate coated with GCC2 antibody. 100 ul of HRP-conjugated reagent was added to all wells, and the plate was covered with a closure plate membrane, and they were reacted at 37° C. for 1 hour. Then, the plate was washed four times using a washing buffer. Then Cromogen solution A and Cromogen solution B were mixed in a 1:1 ratio, and the mixture was injected in 100 ul per well. The reaction was carried out in dark condition at 37° C. for 15 min. 50 ul of stop solution was put into each well, and the concentration of GCC2 protein was confirmed at O.D 450 nm using an ELISA reader within 15 minutes.

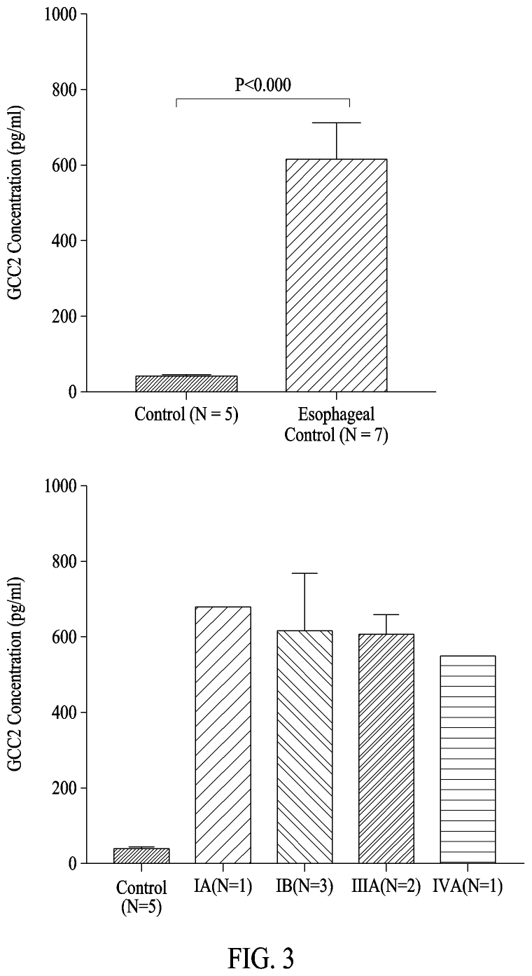

As shown in FIG. 3, the results indicate that the concentration of GCC2 in the exosomes of the esophageal cancer patient group was increased by 12.8 (p<0.000) times compared to the normal group (control). These results suggest that the concentration of GCC2 in exosomes can be usefully used as a diagnostic marker for esophageal cancer.

### Example 3: Cutoff Value of GCC2 Protein in Exosomes for Diagnosis of Esophageal Cancer

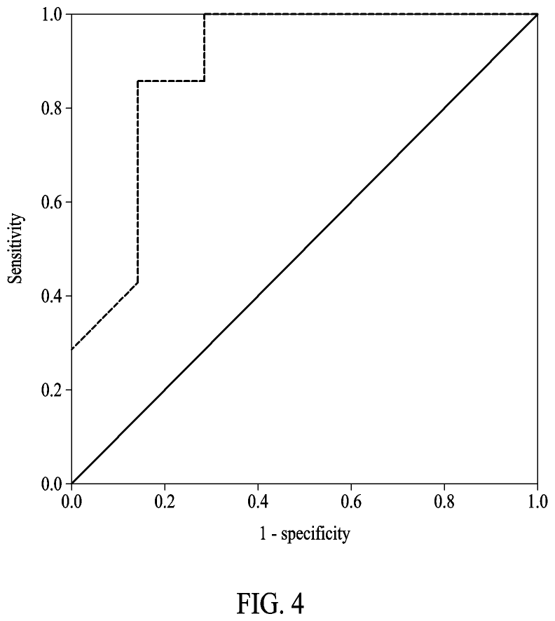

In order to obtain a cutoff value of the GCC2 protein concentration confirmed in Example 2, the ROC curve was comparatively analyzed using a Statistical Package for the Social Sciences (SPSS) program (See FIG. 4). The value obtained from the ROC curve was used to confirm the cutoff value through the formula “specificity=1-(1-specificity).” As a result, it was confirmed to be 521.9695 pg/ml (p value=0.015). The AUC value was 0.888, and the sensitivity and specificity were 85.7%, respectively, confirming that the GCC2 protein in the exosome is useful as a diagnostic marker for esophageal cancer.

### Example 4: Isolation of Exosomes from Thymic Cancer Patient

The plasma of patients with thymic cancer was centrifuged at 4° C. and 5,000×g for 15 minutes to remove debris present in the plasma sample. 500 ul of plasma samples were taken, and exosomes were extracted through size exclusion column chromatography.

### Example 5: Measurement of GCC2 Protein Expression Level in Exosome Derived from Thymic Cancer Patient

In order to confirm whether exosomes containing GCC2 protein are available as markers for diagnosing or predicting prognosis of thymic cancer, ELISA assay was performed using Mybiosource's GRIP and coiled-coil domain containing protein 2 ELISA KIT according to the manufacturer's protocol.

Specifically, two standard samples, two blank samples, and two exosome samples extracted in Example 4 were prepared, and 50 ul each was injected into a 96-well ELISA plate coated with GCC2 antibody. 100 ul of HRP-conjugated reagent was added to all wells, and the plate was covered with a closure plate membrane, and they were reacted at 37° C. for 1 hour. Then, the plate was washed four times using a washing buffer. Then Cromogen solution A and Cromogen solution B were mixed in a 1:1 ratio, and the mixture was injected in 100 ul per well. The reaction was carried out in dark condition at 37° C. for 15 min. 50 ul of stop solution was put into each well, and the concentration of GCC2 protein was confirmed at O.D 450 nm using an ELISA reader within 15 minutes.

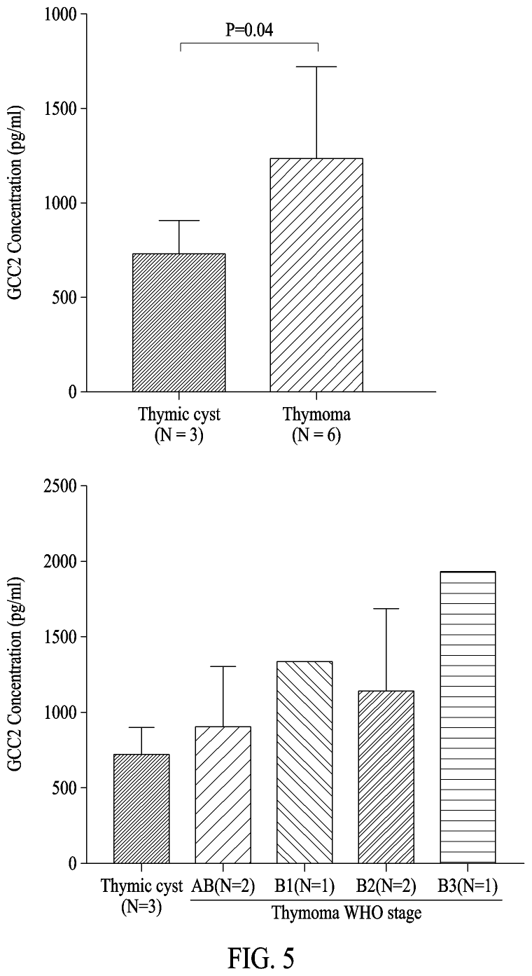

As shown in FIG. 5, the results indicate that the expression of GCC2 was increased in the exosomes of the thymic cancer patient group.

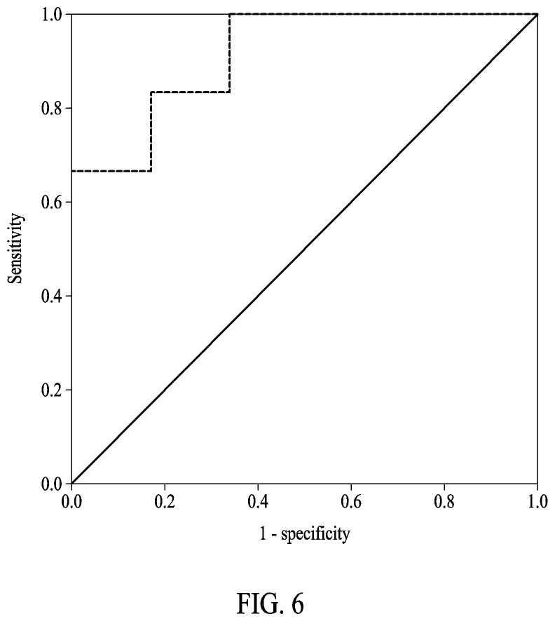

In addition, in order to obtain a cutoff value of the GCC2 protein concentration confirmed above, the ROC curve was comparatively analyzed using a Statistical Package for the Social Sciences (SPSS) program (See FIG. 6). The value obtained from the ROC curve was used to confirm the cutoff value through the formula “specificity=1-(1-specificity).” As a result, it was confirmed to be 909.4513 pg/ml (p value=0.016). The AUC value was 0.917, and the sensitivity and specificity were 83.3%, respectively, confirming that the GCC2 protein in the exosome is useful as a diagnostic marker for thymic cancer.

As described above, although the examples have been described with reference to the limited examples and drawings, various modifications and variations are possible from the above description by those skilled in the art. For example, even if the described techniques are performed in an order different from the described method, and/or the described components are combined or coupled in a different form from the described method, or replaced or substituted by other components or equivalents, appropriate results can be achieved.

Therefore, other implementations, other examples, and equivalents to the claims are also withing the scope of the following claims.

1. A marker composition for diagnosing or predicting prognosis of thymic cancer, the composition comprising an exosome overexpressing a GRIP and coiled-coil domain-containing protein (GCC2) protein.

2. A composition for diagnosing or predicting prognosis of thymic cancer, the composition comprising a primer or probe that specifically binds to a GRIP and coiled-coil domain-containing protein (GCC2) gene in an exosome.

3. A composition for diagnosing or predicting prognosis of thymic cancer, the composition comprising an antibody that specifically binds to a GRIP and coiled-coil domain-containing protein (GCC2) protein in an exosome.

4. A kit for diagnosing or predicting prognosis of thymic cancer, the kit comprising the composition of claim 2.

5. The kit of claim 4, wherein the kit includes at least one selected from the group consisting of an RT-PCR kit, a microarray chip kit, a DNA kit and a protein chip kit.

6.-8. (canceled)

9. A method for providing information necessary for diagnosing thymic cancer or predicting a prognosis thereof, the method comprising steps of:
(a) separating an exosome from a biological sample isolated from a subject; and
(b) measuring an expression level of a GRIP and coiled-coil domain-containing protein (GCC2) gene or protein in the exosome.

10. The method of claim 9, wherein the biological sample includes at least one selected from the group consisting of whole blood, serum, plasma, saliva, urine, sputum, lymph and cells.

11. The method of claim 9, further comprising a step of comparing the measured expression level of the GCC2 gene or protein with a predetermined cutoff value, wherein the predetermined cutoff value is 909.4513 pg/ml.

12. (canceled)

13. A method for screening a therapeutic agent for thymic cancer, the method comprising steps of:
(a) treating a candidate substance for the therapeutic agent for thymic cancer in a biological sample isolated from a patient with thymic cancer;
(b) separating an exosome from the biological sample; and
(c) measuring an expression level of a GRIP and coiled-coil domain-containing protein (GCC2) gene or protein in the exosome.

14. A kit for diagnosing or predicting prognosis of thymic cancer, the kit comprising the composition of claim 3.
